(19) United States
(12) Patent Application Publication
(10) Pub. No.: US 2006/0210528 A1
(43) Pub. Date: Sep. 21, 2006

(54) Therapy for tropical diseases

(75) Inventors: Ian N. Askill, Colorado Springs, CO (US); M. Karen Newell Rogers, Colorado Springs, CO (US); Colin S. Askill, Sketty Swansea (GB)

Correspondence Address: FOLEY & LARDNER LLP, 1530 PAGE MILL ROAD, PALO ALTO, CA, 94304, US

(73) Assignee: Aspire Biotech, Inc.

(21) Appl. No.: 11/437,060

(22) Filed: May 18, 2006

Related U.S. Application Data
(62) Division of application No. 10/259,777, filed on Sep. 30, 2002, now Pat. No. 6,654,305.
(60) Provisional application No. 60/332,752, filed on Nov. 14, 2001.

(51) Int. Cl. A61K 31/765; A61K 31/74

(52) U.S. Cl. 424/78.27

(57) ABSTRACT

Disclosed are methods and formulations for the treatment of topical conditions on mammalian tissues such as skin and mucous tissues mediated at least -in part by viral, bacterial or fungal infections in the mammal. The methods of this invention involve the in situ formation of a polymeric film over the diseased tissue.

### CROSS-REFERENCE TO RELATED APPLICATIONS

This application claims the benefit of U.S. Provisional Patent Application Serial No. 60/332,752 filed Nov. 14, 2001 which application is incorporated herein by reference in its entirety.

### BACKGROUND OF THE INVENTION

### Field of the Invention

This invention is directed to methods and formulations for the treatment of topical conditions on mammalian tissues such as skin and mucous tissues mediated at least in part by viral, bacterial or fungal infections in the mammal. The methods of this invention involve the in situ formation of a polymeric film over the diseased tissue.

In one embodiment, a cure-in-place prepolymeric composition is applied over the diseased tissue to provide for in situ formation of a polymeric film there over. This prepolymeric composition is selected such that the resulting film inhibits atmospheric gas exchange with the diseased tissue which, in turn, weakens the infectious agent. In addition, the polymeric film preferably prevents water loss thereby reducing pain and speeding natural healing of the diseased tissue.

In another embodiment, the polymeric film is formed by solvent casting over the diseased tissue to provide for in situ formation of a polymeric film there over. The resulting film inhibits atmospheric gas exchange with the diseased tissue which, in turn, weakens the infectious agent. In addition, the polymeric film preferably prevents water loss thereby reducing pain and speeding natural healing of the diseased tissue.

The methods and compositions of this invention are especially useful in the treatment of warts and other skin diseases mediated at least in part by a viral infectious agent and, in particular, the papilloma virus.

In one of its composition aspects, the prepolymeric or polymeric composition comprises one or more medicaments in combination therewith. Such medicaments can include an anti-viral agent, anti-fungal agents and/or an ablative agent in, for example, the treatment of warts.

### REFERENCES

The following publications, patent applications and patents are cited in this application as superscript numbers:
¹ Barley, “Methods for Retarding Blister Formation by Use of Cyanoacrylate Adhesives”, U.S. Pat. No. 5,306,490, issued Apr. 26, 1994.
² Barley, et al., Methods for Treating Suturable Wounds by Use of Sutures and Cyanoacrylate Adhesives, U.S. Pat. No. 5,254,132, issued Oct. 19, 1993
³ McIntire, et al., Process for the Preparation of Poly (α-Cyanoacrylates), U.S. Pat. No. 3,654,239, issued Apr. 4, 1972
⁴ Barley, et al., Methods for Treating Non-Suturable Wounds by Use of Cyanoacrylate Adhesives, U.S. Pat. No. 6,342,213, issued Jan. 29, 2002.
⁵ Barley, et al., Methods for Reducing Skin Irritation From Artificial Devices by Use of Cyanoacrylate Adhesives, U.S. Pat. No. 5,653,789, issued Aug. 5, 1997
⁶ Tighe, et al., Methods for Inhibiting Skin Ulceration by Use of Cyanoacrylate Adhesives, U.S. Pat. No. 5,403,591, issued Apr. 4, 1995
⁷ Tighe, et al., for Use of Cyanoacrylates for Providing a Protective Barrier, U.S. Pat. No. 5,580,565, issued Dec. 6, 1996
8 Askill, et al., for Methods for Draping Surgical Incision Sites, U.S. Pat. No. 5,807,563 issued Sep. 15, 1998
⁹ Greff, et al., for Cyanoacrylate Compositions Comprising an Antimicrobial Agent, U.S. Pat. No. 5,684,042, issued Nov. 3, 1997
¹⁰ Askill, et al., for Methods for Draping Surgical Incision Sites Using a Biocompatible Prepolymer, U.S. Pat. No. 5,855,208, issued Jan. 5, 1999
¹¹ Woo, et al., for Gene Therapy for Solid Tumors, Papillomas and Warts, U.S. Pat. No. 6,217,860, issued Apr. 17, 2001
¹² Joyner, et al. for Plasticized Monomeric Adhesive Compositions and Articles Prepared Therefrom, U.S. Pat. No. 2,784,127, issued Mar. 5, 1957
¹³ Columbus, et al. for Adhesive Cvanoacrylate Compositions with Reduced Adhesion to Skin, U.S. Pat. No. 4,444,933, issued Apr. 24, 1984
¹⁴ O'Sullivan, et al. for High Viscosity Cyanoacrylate Adhesive Compositions, and Process for Their Preparation, U.S. Pat. No. 4,038,345, issued Jul. 26, 1977
¹⁵ Roberts, et al. for HPV-Specific Oligonucleotides, U.S. Pat. No. 6,458,940 B2, issued on Oct. 1, 2002.
¹⁶ Otake for Adhesive Container/Feeder, U.S. Pat. No. 4,958,748, issued Sep. 25, 1990
¹⁷ Lee, et al., for Kits Containing Cyanoacrylate Compositions Comprising an Antimicrobial Agent, U.S. Pat. No. 6,090,397, issued Jul. 18, 2000.
¹⁸ Greff, et al. for Compositions for Use in Embolizing Blood Vessels, issued on Sep. 16, 1997.

All of the above publications, patent applications and patents are herein incorporated by reference in their entirety to the same extent as if each individual publication, patent application or patent was specifically and individually indicated to be incorporated by reference in its entirety.

State of the Art

Most topical diseases are treated with steroid creams to reduce the inflammatory response, topical antibiotics, anti-viral or anti-fungal agents to try to kill the infectious agent, or some form of ablative or toxic therapy that destroys skin and hopefully the infectious agent as well.

Numerous mammalian skin conditions, and particularly human skin conditions, are mediated at least in part by bacterial, viral and/or fungal infections. For example, the seminal cause in mammals of the topical skin condition manifesting warts is the papilloma virus;¹⁵ the seminal cause of herpes blisters is the herpes simplex virus; the seminal cause of athlete's foot is the fungus candida. In all cases, the infection manifests itself by unsightly skin lesions/eruptions and in the case of, for example, herpes or athlete's foot eruptions can be particularly painful and infectious.

In the case of warts, current preferred treatments include freezing, surgery, or burning away the wart with electro-cautery, lasers, acid or blistering agents. In a minority of cases, immuno-stimulants will be used to facilitate an anti-viral immune response against the wart.

Typical of the state of the art is the use of a composition commercially available under the tradename “Compound W”, which contains 17% salicylic acid. This composition, which is applied daily, dissolves the wart slowly but will also dissolve adjacent skin and so must be used carefully. In addition, the use of gene therapy in the treatment of papilloma viral infections has been disclosed in U.S. Pat. No. 6,217,860.¹¹ However, to date, such gene treatment has not been commercialized.

This invention is directed to the novel and unexpected discovery that these skin conditions can be treated by forming a polymeric film over the diseased tissue. This film inhibits atmospheric gas exchange with the diseased tissue which, in turn, weakens the infectious agent. In addition, the polymeric film preferably prevents water loss thereby reducing pain and speeding natural healing of the diseased tissue.

In a particularly preferred embodiment, the polymeric film is formed from a cyanoacrylate prepolymer although other prepolymeric compositions can also be used.

Heretofore, prepolymeric cyanoacrylate compositions have been disclosed for use in a variety of medical environments such as an alternative or adjunct to sutures² or as a hemostat³. Other described uses of cyanoacrylate prepolymers include their use on mannnalian tissue to form polymeric films which are utilized:

to prevent friction blister formation,¹

in treating small non-suturable wounds,⁴

in inhibiting surface skin irritation arising from friction between the skin surface and artificial devices such as tapes, prosthetic devices, casts, etc.,⁵

as surgical incise drapes,⁸

in inhibiting skin ulceration,⁶ and

forming a protective film to inhibit skin degradation due to incontinence.⁷

Similarly, Askill, et al.,¹⁰ discloses that biocompatible prepolymeric compositions, in addition to cyanoacrylates, can be used to form in situ surgical drapes.

However, there is no disclosure of utilizing in situ formed polymeric films on mammalian skin to treat skin conditions mediated at least in part by infectious agents.

### SUMMARY OF THE INVENTION

This invention provides for the use of cure-in-place barrier films with improved gas exchange barrier properties. These films are formed in place over skin or mucous membrane lesions which lesions are formed, at least in part, by bacterial, viral or fungal infectious agents. Without being limited to any theory, it is believed that the robustness and resistance to healing of many of these topical diseases is due to their position on the skin or mucous surface of the infected mammal which allows them to absorb gases from and release gases to the atmosphere. That is to say that infectious agents responsible for forming such topical diseases either require oxygen and/or release of generated gases to proliferate. Again without being limited to any theory, it is believed that the barrier films formed in situ on mammalian skin inhibit atmospheric gas exchange thereby inhibiting proliferation of the infectious agent. In turn, this will lead to speedier healing of the lesions.

Accordingly, in one of its method aspects, this invention is directed to a method for treating skin or mucous membrane lesions in a mammal wherein the formation of said lesions is mediated at least in part by one or more bacterial, viral and/or fungal agents which method comprises:

(a) identifying skin or mucous membrane lesion(s) in a mammal wherein the formation of said lesions is mediated at least in part by one or more bacterial, viral and/or fungal infectious agents; and

(b) forming a polymeric film over said lesion(s) which inhibits proliferation of said infectious agents in said lesion(s).

In one embodiment, the polymeric film is formed by applying to the lesion a sufficient amount of a biocompatible prepolymeric composition under conditions wherein a polymeric film is formed in situ over said lesion(s).

Preferably, the biocompatible prepolymeric composition comprises a polymerizable biocompatible prepolymer which is selected from the group of polymerizable prepolymers consisting of urethane acrylate, cyanoacrylate esters, (C₁-C₆ alkyl) methacrylate esters, (C₁-C₆ alkyl) acrylate esters, (C₁-C₆ hydroxyalkyl) acrylate esters, (C₁-C₆ hydroxyalkyl) alkacrylate esters, silicone, styrene, α-methyl styrene, vinyl acetate, one and two component epoxy materials, mixtures thereof, and the like.

More preferably, the polymerizable biocompatible prepolymer is a cyanoacrylate ester prepolymer which, in monomeric form, is represented by formula I: 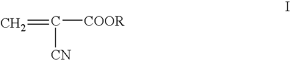
where R is selected from the group consisting of:

alkyl of 1 to 10 carbon atoms,

alkenyl of 2 to 10 carbon atoms,

cycloalkyl groups of from 5 to 8 carbon atoms,

phenyl,

—R¹—O—R² where R¹ is alkylene of from 2 to 6 carbon atoms and R² is alkyl of from 1 to 6 carbon atoms (preferably, 2-ethoxyethylene, 3-methoxybutylene, or 3-propoxypropylene),

and a substituent of the formula: 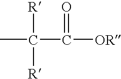
wherein each R′ is independently selected from the group consisting of:
hydrogen and methyl, and

R″ is selected from the group consisting of:
alkyl of from 1 to 6 carbon atoms,
alkenyl of from 2 to 6 carbon atoms,
alkynyl of from 2 to 6 carbon atoms,
cycloalkyl of from 3 to 8 carbon atoms,
aralkyl selected from the group consisting of benzyl, methylbenzyl and phenylethyl,
phenyl, and
phenyl substituted with 1 to 3 substituents selected from the group consisting of hydroxy, chloro, bromo, nitro, alkyl of 1 to 4 carbon atoms, and alkoxy of from 1 to 4 carbon atoms.

In one preferred embodiment, R is alkyl of from 2 to 10 carbon atoms and more preferably alkyl of from 2 to 8 carbon atoms. Even more preferably, R is butyl, pentyl or octyl and most preferably, R is n-butyl.

In another preferred embodiment, R is —R¹—O—R² where R¹ is alkylene of from 2 to 6 carbon atoms and R² is alkyl of from 1 to 6 carbon atoms. Even more preferably, R is selected from the group consisting of ethoxyethylene, methoxybutylene, and propoxypropylene.

In another embodiment, the polymeric film is formed in situ by applying to the lesion a sufficient amount of a biocompatible polymeric composition comprising a biocompatible solvent and a biocompatible polymer dissolved therein under conditions wherein a polymeric film is formed in situ over said lesion upon dissipation of the solvent.

Preferably, the biocompatible polymer is selected from the group of polymers consisting of urethane acrylate polymers, cyanoacrylate ester polymers, (C₁-C₆ alkyl) methacrylate ester polymers, (C₁-C₆ alkyl) acrylate ester polymers, (C₁-C₆ hydroxyalkyl) acrylate ester polymers, (C₁-C₆ hydroxyalkyl) alkacrylate ester polymers, silicone polymers, styrene polymers, α-methyl styrene polymers, vinyl acetate-polymers, vinyl alcohol polymers, one and two component epoxy materials, copolymers and mixtures thereof, and the like.

In another preferred embodiment, the in situ formed polymeric film has a thickness of no more than about 1 millimeter and, more preferably, the polymer layer has a thickness of from about 2 to about 500 microns and still more preferably from about 50 to about 200 microns.

In still another preferred embodiment, the polymeric film, whether formed by in situ polymerization of the polymerizable monomer or by solvent casting a solution of polymer dissolved in a biocompatible solvent, inhibits atmospheric gas exchange with the lesion by at least 30%; preferably by at least 50%; more preferably by at least 75 %; and most preferably by at least 90% as compared to the amount of atmospheric gas exchanged with similar lesions in the absence of the polymeric film.

In one of its composition aspects, this invention is directed to a biocompatible composition comprising:

a polymer film forming component selected from the group consisting of biocompatible prepolymers and biocompatible polymer; and

a gas retarding agent.

In another of its composition aspects, this invention is directed to a biocompatible composition comprising:

a polymer film forming component selected from the group consisting of biocompatible prepolymers and biocompatible polymer; and

an effective amount of an anti-infectious agent selected from the group consisting of anti-fungal and anti-viral medicaments.

In still another of its composition aspects, this invention is directed to a biocompatible composition comprising:

a polymer film forming component selected from the group consisting of biocompatible prepolymers and biocompatible polymer; and

an effective amount of an ablative agent.

### DETAILED DESCRIPTION OF THE PREFERRED EMBODIMENTS

This invention is directed to methods and formulations for the treatment of topical infections on mammalian tissue. However, prior to discussing this invention in further detail, the following terms will first be defined.

Definitions

As used herein, the following terms have the following meanings:

The term “polymerizable biocompatible prepolymer compositions” refer to compositions comprising polymerizable monomers, oligomers or mixtures thereof including single or multi-component systems. The prepolymer composition will polymerize in situ on mammalian skin to form an adherent, water-insoluble polymeric layer over the skin. The prepolymer and resulting polymeric film are biocompatible with the skin as measured by the lack of moderate to severe skin irritation and the resulting polymer film is substantially non-toxic and can be removed from the skin by conventional means, e.g., sloughing off with the epidermal layer of the skin or the diseased tissue.

Included within the term “polymerizable biocompatible prepolymer compositions” are both single and multi-component systems. Single component prepolymer compositions include those wherein a single prepolymer is capable of polymerizing under suitable polymerization conditions (e.g., free radical conditions) to provide for a polymer film on mammalian skin. Such single component systems include well known reactive vinyl groups which form a biocompatible polymer such as cyanoacrylate esters, urethane acrylate esters, (C₁-C₆ alkyl) methacrylate esters, (C₁-C₆ alkyl) acrylate esters, (C₁-C₆ hydroxyalkyl) acrylate esters, (C₁-C₆ hydroxyalkyl) alkacrylate esters, silicone, styrene, α-methyl styrene, vinyl acetate, and the like. Additionally, such single component systems can also comprise conventional polymerization inhibitors, polymerization initiators, colorants, perfumes, etc.

Multi-component prepolymer compositions include those wherein two or more components are employed to co-react under suitable polymerization conditions to provide for a polymer film on mammalian skin. An example of a two component system is a diepoxide and a diamine specifically exemplified by bis-phenol A diglycidyl ether and ethylene diamine.

Preferred prepolymers for use in this invention include, by way of example only, cyanoacrylate esters, urethane acrylate esters, (C₁-C₆ alkyl) methacrylate esters, (C₁-C₆ alkyl) acrylate esters, (C₁-C₆ hydroxyalkyl) acrylate esters, (C₁-C₆ hydroxyalkyl) alkacrylate esters, styrene, α-methyl styrene, vinyl acetate esters, one and two component epoxy materials, mixtures thereof, and the like. Mixtures of such prepolymers can also be employed.

A particularly preferred prepolymer is a “polymerizable cyanoacrylate ester” which refers to polymerizable formulations comprising cyanoacrylate monomers or polymerizable oligomers which, in their monomeric form, are preferably compounds represented by formula I as described above.

More preferably, in formula I, R is an alkyl group of from 2 to 10 carbon atoms including ethyl, n-propyl, iso-propyl, n-butyl, iso-butyl, sec-butyl, n-pentyl, iso-pentyl, n-hexyl, iso-hexyl, 2-ethylhexyl, n-heptyl, octyl, nonyl, and decyl. More preferably, R is butyl, pentyl or octyl and most preferably, R is n-butyl. Mixtures of such compounds can also be employed as disclosed by Berger, et al., U.S. Pat. No. 5,998,472 which is incorporated herein by reference in its entirety.

In another preferred embodiment, in formula I, R is —R¹—O—R² where R¹ is alkylene of from 2 to 6 carbon atoms and R² is alkyl of from 1 to 6 carbon atoms. Even more preferably, R is selected from the group consisting of ethoxyethylene, methoxybutylene; and propoxypropylene.

Preferred cyanoacrylate esters for use in the invention include 3-propoxypropyl-2-cyanoacrylate and n-butyl-2-cyanoacrylate.

The polymerizable cyanoacrylate esters described herein rapidly polymerize in the presence of water vapor or tissue protein, and the n-butyl-cyanoacrylate bonds to mammalian skin tissue without causing histotoxicity or cytotoxicity.

Such polymerizable cyanoacrylate esters are sometimes referred to herein as prepolymers and compositions comprising such esters are sometimes referred to herein as prepolymer compositions.

Prepolymers suitable for use in this invention are well known in the art and are described in, for example, U.S. Pat. Nos. 3,527,224; 3,591,676; 3,667,472; 3,995,641; 4,035,334; 4,650,826; and 5,855,208; the disclosures of each are incorporated herein by reference in their entirety.

The term “biocompatible polymer compositions” refer to compositions comprising a biocompatible polymer, preferably dissolved in a biocompatible solvent such that when applied to mammalian tissue, the solvent dissipates leaving a polymeric film over the tissue to which the composition was applied. The polymer and solvent are biocompatible with the skin as measured by the lack of moderate to severe skin irritation and the resulting polymer film is substantially non-toxic and can be removed from the skin by conventional means, e.g., sloughing off with the epidermal layer of the skin or the diseased tissue.

Preferred polymers for use in this invention include, by way of example only, polymers obtained from cyanoacrylate esters, urethane acrylate esters, (C₁-C₆ alkyl) methacrylate esters, (C₁-C₆ alkyl) acrylate esters, (C₁-C₆ hydroxyalkyl) acrylate esters, (C₁-C₆ hydroxyalkyl) alkacrylate esters, styrene, α-methyl styrene, vinyl acetate esters (including hydrolysis products thereof), one and two component epoxy materials, copolymers and mixtures thereof, and the like.

The polymer composition comprises a biocompatible polymer in an amount from about 5 weight percent to about 60 weight percent of the composition, more preferably from 15 weight percent to about 45 weight percent of the composition.

The biocompatible polymer is preferably characterized as having a molecular weight from about 10,000 Daltons to about 1,500,000 Daltons and is selected to yield solutions of appropriate viscosity.

The biocompatible solvent preferably includes dimethylsulfoxide, tetrahydrofuran, ethanol, acetone, methyl ethyl ketone and esters such as ethyl acetate. Such solvents are characterized by their high vapor pressure such that once the composition is applied to the skin, the solvent quickly dissipates (less than 5 minutes) leaving a durable, flexible film.

The term “biocompatible plasticizer” refers to any material which is soluble or dispersible in the prepolymer or polymer composition, which increases the flexibility of the resulting polymeric film coating on the skin surface, and which, in the amounts employed, is compatible with the skin as measured by the lack of moderate to severe skin irritation. Suitable plasticizers are well known in the art and include those disclosed in U.S. Pat. Nos. 2,784,127¹² and 4,444,933¹³ the disclosures of both of which are incorporated herein by reference in their entirety. Specific plasticizers include, by way of example only, acetyl tri-n-butyl citrate, acetyl trihexyl citrate, butyl benzyl phthalate, dibutyl phthalate, dioctylphthalate, n-butyryl tri-n-hexyl citrate, diethylene glycol dibenzoate, and the like. The particular biocompatible plasticizer employed is not critical and preferred plasticizers include dioctylphthalate and C₂-C₄-acyl tri-n-hexyl citrates.

The term “thickening agent” refers to any biocompatible material which increases the viscosity of the composition. Suitable thickening agents include, by way of example, polymethyl methacrylate (PMMA) or other preformed polymers soluble or dispersible in the composition, a suspending agent such as fumed silica and the like with PMMA being preferred. Fumed and modified fumed silica are particularly useful in producing a gel for topical application having a viscosity of from about 1,500 to about 1,000,000 centipoise at 20° C. Suitable thickening agents for the compositions described herein also include a partial polymer of the alkyl cyanoacrylate as disclosed in U.S. Pat. Nos. 3,654,239³ and 4,038,345¹⁴ both of which are incorporated herein by reference in their entirety.

Thickening agents are deemed to be biocompatible if they are soluble or dispersible in the composition and are compatible with the skin as measured by the lack of moderate to severe skin irritation.

The term “anti-fungal agent” refers to any of a number of well known anti-fungal agents including, for example, butenafine, naftifine, terbinafine, bifonazole, butoconazole, chlordantoin, chlormidazole, cloconazole, cimetidine, clotrimazole, econazole, enilconazole, fenticonazole, flutrimazole, isoconazole, ketoconazole, lanoconazole, miconazole, omoconazole, oxiconazole nitrate, sertaxonazole, sulconazole, tioconazole, tolciclate, tolindate, tolnaftate, acrisorcin, amorolfme, biphenamine, bromocalicylchloranilide, buclosamide, calcium propionate, chlorphenesin, ciclopirox, cloxyquin, coparaffinate, diamthazole dihydrochioride, exalamide, flucytosine, halethazole, hexetidine, loflucarban, nifuratel, potassium iodide, propionic acid, pyrithione, salicylanilide, sodium propionate, sulbentine, tenonitrozole, triacetin, ujothion, undecylenic acid, and zinc propionate. Each of these are well known in the art as anti-fungal agents and are described in the 12^{th} Edition of the Merck Index (1996).

The term “anti-viral agent” refers to any of a number of well known anti-viral agents including, by way of example only, acyclovir, cidofovir, cytarabine, dideoxyadenosine, didanosine, docosanol, edoxudine, famciclovir, floxuridine, ganciclovir, idoxuridine, inosine pranobex, lamirudine, MADU, penciclovir, sorivudine, stavudine, trifluridine, valacyclovir, vidarabine, zalcitabine, zidovudine, acemannan, acetylleucine monoethanolamine, amantadine, amidinomycin, delavirdine, foscarnet sodium, indinavir, interferon-α, interferon-β, interferon-γ, kethoxal, methisazine, moroxydine, nevirapine, podophyllo-toxin, ribavirim, rimantadine, ritonavir, saquinavir, statolon, tromantadine, and xenozoic acid. Each of these are well known in the art as anti-viral agents and are described in the 12^{th} Edition of the Merck Index (1996).

The term “ablative agent” refers to well known materials which facilitate skin removal. Such agents include, by way of example only, salicylic acid, acetic acid, lactic acid, trichloroacetic acid, cantharadin, and the like.

The term “gas retarding agent” refers to any component which when added to the polymer film-forming composition enhances the gas retarding capacity of the resulting polymeric film. A particularly preferred component includes biocompatible polymers comprising vinyl alcohol including homopolymers, copolymers, terpolymers of vinyl alcohol and the like. Biocompatible copolymers comprising vinylalcohol include the commercially available ethylene vinyl alcohol (EVOH) and its use in vivo is described in U.S. Pat. No. 5,667,767¹⁸ which is incorporated herein by reference in its entirety. In addition, metal particles, such as aluminum, silver, gold and the like, when added to the polymer film will enhance the gas retarding capacity of the film.

The term “solvent casting” refers to the technique wherein a preformed biocompatible polymer, dissolved in a biocompatible solvent, is applied to the tissue and the solvent is allowed to dissipate thereby leaving a polymeric film on the tissue.

Methods

The methods of this invention comprise the in situ formation of a polymer film on diseased mammalian tissue.

The treatment protocol preferably involves tissue preparation prior to in situ formation of the polymer film. For example, the lesion is first conventionally treated by the attending health care professional by cleaning with an appropriate antimicrobial composition. The lesion is preferably dried, e.g., blotted dry, and then an adherent polymeric film is formed there over.

In the case of prepolymer, a sufficient amount of a biocompatible, polymerizable prepolymer composition is applied to the lesion such that, upon contact with the lesion, the prepolymer polymerizes in situ to form a polymeric film.

Polymerization occurs at ambient conditions for a sufficient period of time to allow robust films to form. In general, the particular length of time required for polymerization will vary depending on factors such as the type and amount of prepolymer applied, the temperature of the tissue, the moisture content of the tissue, the surface area of tissue, and the like. However, in a preferred embodiment, polymerization is generally complete within about 10 to about 100 seconds while the tissue is maintained at ambient conditions; however, in some cases, polymerization can occur up to about 5 minutes. During this period, the tissue is maintained in a position which permits the prepolymer to polymerize and form a polymeric film while minimizing any movement which might dislodge the prepolymer from the tissue or create undesirable bonding.

Alternatively, the polymeric film can be formed by solvent casting which procedures preferably entails the use of a composition comprising both a biocompatible polymer and a biocompatible solvent in which the polymer is dissolved. The composition is applied onto the tissue whereupon the solvent dissipates (e.g., evaporates and/or passes through the skin barrier) leaving a polymeric film over the tissue.

Sufficient amounts of the composition are employed to cover (i.e., coat) the entire tissue site with a layer of polymer. If necessary, excess prepolymer or polymer composition can be removed with a wipe or tissue paper before polymerization or before solvent dissipation.

The resulting polymeric film acts as a barrier film which strongly adheres to the skin, is flexible and waterproof. Such strong adherence effectively eliminates the possibility that the film will separate from the tissue. In the case of application to mammalian skin, the polymeric film will only adhere to the skin for a period of about 1-4 days after which time it sloughs off. Diseased tissue such as wart columns may replicate more slowly allowing the therapeutic effect to be prolonged. This occurs because the polymer adheres only to the epidermal layer which is continuously in the process of being sloughed off and replaced by the underlying cells. Accordingly, the polymer film need not be removed from such skin or diseased tissue.

The polymeric film should be maintained in an unbroken manner over the entire tissue. This can be assured by careful application of the prepolymer or polymer composition onto the tissue. Additionally, the use of a plasticizer in either of these compositions will facilitate the maintenance of the polymeric film in an unbroken manner and will inhibit cracking of the film.

In one embodiment, after application of the initial polymeric layer, a second, preferably thinner, layer is applied thereto. Additional polymer layers can be formed as needed to maintain an unbroken coating covering over the tissue.

Application is conducted under conditions wherein the polymeric film preferably has a thickness of no more than about 1 millimeter and, more preferably, the polymer layer has a thickness of from about 2 to about 500 microns and still more preferably from about 50 to about 200 microns. The amount of composition applied to a unit area to obtain such thicknesses is well within the skill of the art.

The size and thickness of the polymeric film formed onto the tissue area can be readily controlled by the amount and viscosity of prepolymeric or polymeric composition packaged in a single dose product or by use of a multiple use dispenser which governs the amount of material applied onto a unit area of surface skin. In this regard, the dispenser described by Otake, U.S. Pat. No. 4,958,748,¹⁶ which is incorporated by reference in its entirety, is one example of a dispenser which dispenses the composition in a controlled dropwise manner. Other methods for the controlled dispersement of the prepolymeric or polymeric composition include, by way of example, a spray applicator, brush, wipe, swab or solid paddle applicator, applicators for repeated and intermittent use of the composition and the like.

In applicators, the composition is stored at ambient conditions and can be provided in sterile form.

Compositions

The biocompatible polymer or prepolymer compositions comprising the polymerizable prepolymers are prepared by conventional methods of mixing the appropriate components until homogenous.

The specific viscosity of these compositions depends, in part, on the intended application of the composition. For example, relatively low viscosities are often preferred where application is to be made to a large surface area (e.g., diseased tissue between toes). This preference results from the fact that those forms are less viscous and, accordingly, will permit more facile large surface area application of a thin application. Contrarily, where application is to be made to a specific position on the skin (e.g., warts), higher viscosity materials are preferred to prevent “running” of the material to unintended locations.

Accordingly, these compositions have a viscosity of from about 2 to 50,000 centipoise at 20° C. Preferably the less viscous compositions have a viscosity of from about 2 to 1,500 centipoise at 20° C.

In the case of prepolymeric compositions, the biocompatible prepolymer preferably employed in these compositions is almost entirely in monomeric form and the composition has a viscosity of from about 2 to about 500 centipoise at 20° C.

A thickening agent is optionally employed to increase the viscosity of the either the polymeric or prepolymeric composition which thickening agent is any biocompatible material which increases the viscosity of the composition. Suitable thickening agents include, by way of example, polymethyl methacrylate (PMMA) or other preformed polymers soluble or dispersible in the composition, a suspending agent such as fumed silica and the like, with PMMA being preferred. Fumed silica is particularly useful in producing a gel for topical application having a viscosity of from about 1500 to 50,000.

Thickening agents are deemed to be biocompatible if they are soluble or dispersible in the composition and are compatible with the skin as measured by the lack of moderate to severe skin irritation.

The compositions described herein may optionally include a biocompatible plasticizer and such plasticizers are preferably included in the composition from about 10 to 40 weight percent and more preferably from about 20 to 30 weight percent based on the total weight of the composition.

Additionally, the prepolymer compositions described herein may preferably include a polymerization inhibitor and may include a polymerization initiator in effective amounts to provide for in situ polymerization on mammalian skin. For example, an effective amount of a polymerization inhibitor is preferably included in the composition to inhibit premature polymerization of the composition. Likewise, for non-ionic polymerization, a polymerization initiator is included in the composition in effective amounts to initiate polymerization when the composition is placed under polymerization conditions (e.g., light). As above, such initiators include thermal initiators, light activated initiators and the like and in situ polymerization of the prepolymer composition on mammalian skin preferably occurs within 0.5 to 5 minutes.

The polymeric or prepolymeric compositions described herein may additionally contain one or more optional additives such as colorants, perfumes, rubber modifiers, modifying agents, etc. In practice, each of these optional additives should be both miscible and compatible with the biocompatible prepolymer composition and compatible with the resulting polymer. Compatible additives are those that do not prevent the use of the biocompatible prepolymers in the manner described herein.

In general, colorants are added so that the polymer layer formed on the skin will contain a discrete and discernable color. Perfumes are added to provide a pleasant smell to the formulation. Rubber modifiers are added to further enhance the flexibility of the resulting polymer layer. The amount of each of these optional additives employed in the composition is an amount necessary to achieve the desired effect.

In addition, the prepolymeric and polymeric compositions described herein may include an effective amount of one or more of the following:

an anti-viral and/or anti-fungal agent to further facilitate inhibition of infectious viral and/or fungal agents;

an ablative agent to facilitate removal of the lesion; and

a gas retarding agent to enhance the gas retarding capacity of the resulting polymeric film.

Preferably, when used in a prepolymeric composition, each of these components are selected to be compatible with the prepolymer which compatibility is determined by not effecting premature polymerization of the prepolymer; not preventing in situ polymerization of the prepolymer when applied to mammalian tissue; and permits the formation of a flexible, durable film.

On the other hand, incompatible components can still be used provided that they are employed in a two-component system such as described by Lee, et al., U.S. Pat. No. 6,090,397¹⁷ which patent is incorporated herein by reference in its entirety.

Still further, for components which are insoluble in either the polymeric or prepolymeric composition, a solvent or co-solvent can be employed to solubilize this component. The solvent or co-solvent is selected to be biocompatible and, when so used, can enhance the adherence of the resulting film to the diseased tissue.

The methods and compositions of this invention are especially useful in the treatment of warts and other skin diseases mediated at least in part by a viral infectious agent and, in particular, the papilloma virus. When so employed, the polymeric or prepolymeric compositions are applied topically to the wart in an amount sufficient to cover the wart. In addition to the prepolymer or polymer component, the compositions can further comprise an effective amount of an ablative agent such as salicylic acid to facilitate removal of the wart.

Utility

The methods and compositions described herein are useful in forming in situ polymeric films to treat skin conditions on a mammal partially mediated by bacterial, viral, and/or fungal infectious agents. The methods of this invention are particularly usefuil for treatment of the papilloma virus. Further, the polymeric film has utility by weakening the infectious agent thereby facilitating healing. Still further, the polymeric film prevents water loss thereby reducing the pain and speeding natural healing of the disease.

### EXAMPLES

The following examples illustrate how the methods of this invention could be used.

### Example 1

A patient presents to a dermatologist with two similar plantar warts on the soles of her left and right feet. The wart on the right foot is treated by freezing with liquid nitrogen. The wart on the left foot is treated with a liquid composition comprising propoxypropyl cyanoacrylate containing 10% of polyvinyl alcohol-ethylene copolymer and the patient is given more solution to continue applying the barrier film every 3-4 days. The freeze burn on the right makes walking on the foot painful for several days. The wart on the right foot appears to have resolved after 3 weeks but has returned after two months. The polymeric film on the left foot reduces the pain from the plantar wart and the wart resolves in 4 weeks. Treatment of the sites continues for two weeks after resolution and the wart does not return.

### Example 2

A 32-year old female with two cold sores on her upper lip and one on the lower lip treats one with an over the counter cold sore remedy, another with ethoxyethyl cyanoacrylate containing 30% low molecular weight polyvinyl alcohol and the third with propoxypropyl cyanoacrylate containing 5 % docosanol. The cold sore treated with propoxypropyl cyanoacrylate containing 5 % docosanol resolves in 4 days. The cold sore treated with ethoxyethyl cyanoacrylate containing 30% low molecular weight polyvinyl alcohol resolved in 5 days and the cold sore treated with the over the counter cold sore remedy resolved on 7 days.

What is claimed is:

1. A method for treating a skin or mucous membrane lesion in a mammal wherein the formnation of the lesion is mediated at least in part by one or more bacterial, viral and/or fungal agents which method comprises applying to the lesion a sufficient amount of a composition comprising a polymerizable, biocompatible prepolymer and a gas retarding agent under conditions wherein the composition forms a polymeric film in situ over the lesion which inhibits proliferation of the infectious agents in the lesion.

2. The method of claim 1, wherein the prepolymer is selected from the group consisting of urethane acrylate, cyanoacrylate esters, (C₁-C₆ alkyl) methacrylate esters, (C₁-C₆ alkyl) acrylate esters, (C₁-C₆ hydroxyalkyl) acrylate esters, (C₁-C₆ hydroxyalkyl) alkacrylate esters, silicone, styrene, α-methyl styrene, vinyl acetate, one and two component epoxy materials and mixtures thereof.

3. The method of claim 2 wherein the polymerizable biocompatible prepolymer is a cyanoacrylate ester prepolymer.

4. The method of claim 3 wherein the cyanoacrylate ester prepolymer, in monomeric form, is represented by formula I: 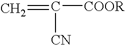
where R is selected from the group consisting of:
alkyl of 1 to 10 carbon atoms,
alkenyl of 2 to 10 carbon atoms,
cycloalkyl groups of from 5 to 8 carbon atoms,
phenyl,
—R¹—O—R² where R¹ is alkylene of from 2 to 6 carbon atoms and R² is alkyl of from 1 to 6 carbon atoms,
and a substituent of the formula: 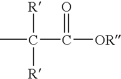
wherein each R′ is independently selected from the group consisting of:
hydrogen and methyl, and
R″ is selected from the group consisting of:
alkyl of from 1 to 6 carbon atoms,
alkenyl of from 2 to 6 carbon atoms,
alkynyl of from 2 to 6 carbon atoms,
cycloalkyl of from 3 to 8 carbon atoms,
aralkyl selected from the group consisting of benzyl, methylbenzyl and phenylethyl,
phenyl, and
phenyl substituted with 1 to 3 substituents selected from the group consisting of hydroxy, chloro, bromo, nitro, alkyl of 1 to 4 carbon atoms, and alkoxy of from 1 to 4 carbon atoms.

5. The method according to claim 4 wherein R is selected from the group consisting of alkyl of from 2 to 10 carbon atoms and —R¹—O—R² where R¹ is alkylene of from 2 to 6 carbon atoms and R² is alkyl of from 1 to 6 carbon atoms.

6. The method according to claim 5 wherein R is alkyl of 2 to 10 carbon atoms.

7. The method according to claim 6 wherein R is alkyl of 2 to 8 carbon atoms.

8. The method according to claim 7 wherein R is selected from the group consisting of butyl, pentyl and octyl.

9. The method according to claim 8 wherein R is n-butyl.

10. The method according to claim 5 wherein R is —R¹—O—R^{2.}

11. The method according to claim 10 wherein —R¹—O—R² is selected from the group consisting of ethoxyethylene, propoxypropylene and methoxybutylene.

12. The method according to claim I wherein the gas retarding agent is a biocompatible polymer comprising polyvinyl alcohol.

13. The method according to claim 12 wherein the biocompatible polymer comprises an ethylene vinyl alcohol copolymer.

14. The method according to claim 1 wherein the gas retarding agent comprises metal particles.

15. The method according to claim 1 wherein the in situ formed polymeric film inhibits atmospheric gas exchange with the lesion by at least 30% as compared to the amount of atmospheric gas exchanged with similar lesions in the absence of the polymeric film.

16. The method according to claim 15 wherein the in situ formed polymeric film inhibits atmospheric gas exchange with the lesion by at least 50% as compared to the amount of atmospheric gas exchanged with similar lesions in the absence of the polymeric film.

17. The method according to claim 16 wherein the in situ formed polymeric film inhibits atmospheric gas exchange with the lesion by at least 75% as compared to the amount of atmospheric gas exchanged with similar lesions in the absence of the polymeric film.

18. The method according to claim 17 wherein the in situ formed polymeric film inhibits atmospheric gas exchange with the lesion by at least 90% as compared to the amount of atmospheric gas exchanged with similar lesions in the absence of the polymeric film.

19. The method according to claim 1 wherein the in situ formed polymeric films inhibits oxygen exchange between the lesion and the atmosphere.

20. The method according to claim 1 wherein the in situ formed polymeric film further comprises one or more of an anti-viral agent, an anti-fungal agent or an ablative agent.
